(19) United States
(12) Patent Application Publication
(10) Pub. No.: US 2003/0065020 A1
(43) Pub. Date: Apr. 3, 2003

(54) Treatment of macular degeneration

(76) Inventors: Catharine Gale, Southampton (GB); Nigel Hall, Southampton (GB); Christopher Martyn, Southampton (GB); David Phillips, Southampton (GB)

Correspondence Address: PALMER & DODGE, LLP, KATHLEEN M. WILLIAMS, 111 HUNTINGTON AVENUE, BOSTON, MA, 02199, US

(21) Appl. No.: 10/194,119

(22) Filed: Jul. 12, 2002

Related U.S. Application Data
(60) Provisional application No. 60/305,276, filed on Jul. 13, 2001.

(51) Int. Cl. A61K 31/401; A61K 31/366; A61K 31/255

(52) U.S. Cl. 514/423; 514/460; 514/548

(57) ABSTRACT

We describe a method of treating or preventing macular degeneration in a patient, the method comprising administering an HMG-CoA reductase inhibitor to the patient.

### PRIORITY

This application claims priority under 35 U.S.C. §119(e) to U.S. Provisional Application Serial No. 60/305,276, filed Jul. 13, 2001, the contents of which are incorporated herein in their entirety.

### FIELD OF THE INVENTION

This invention relates to treatment of eye diseases, in particular, degenerative diseases of the retina.

### BACKGROUND OF THE INVENTION

Age-related macular degeneration (AMD) is a degenerative disease that affects the macula, a small spot in the central area of the retina located at the back of the eye responsible for sight in the centre of the field of vision. Symptoms of macular degeneration include straight lines in the field of vision appear wavy; type in books, magazines and newspapers appears blurry; and dark or empty spaces block the centre of vision.

Age-related macular degeneration is the commonest cause of blindness in developed countries. For example, in the United States, macular degeneration affects over 13 million people, and is the leading cause of visual impairment for persons age 75 and older (30% affected). It is also the most common cause of new cases of visual impairment among those over age 65, with at least 10% of individuals having lost some central vision as a result of macular degeneration. Age-related macular degeneration is also the commonest cause of blindness in the United Kingdom. A tendency to develop macular degeneration may be seen in some families because of genetic factors. Although macular degeneration almost never results in complete blindness since side vision is usually not affected, when a substantial amount of central vision is lost, people with macular degeneration may find it very difficult to do simple, everyday activities that require sharp vision.

Macular degeneration occurs in two forms, “atrophic” (non-exudative) and “exudative” (commonly known as “dry” form and “wet” form respectively). The causes of “dry” macular degeneration, in which there is a slow breakdown of light-sensing cells in the macula, subsequently reducing central vision, are at present unclear. About 90 percent of people with macular degeneration have this type of the disease. There is as yet no treatment for “dry” macular degeneration, although it has been suggested that supplemental vitamins and minerals may slow the progress of the disease. As “dry” macular degeneration worsens, new, fragile blood vessels grow beneath the macula. These new blood vessels often leak blood and fluid, causing rapid damage to the macula and quickly leading to loss of central vision; this form of the disease is known as “wet” macular degeneration. Although only 10 percent of those with macular degeneration have this type of the disease, “wet” macular degeneration accounts for 90 percent of all blindness resulting from macular degeneration. Treatment consists of laser surgery to destroy new blood vessels is used to treat some cases of the “wet” form of macular degeneration. Photodynamic therapy may also be employed

However, only about 15 percent of patients with the “wet” form of macular degeneration are eligible for laser surgery because the new blood vessels may have advanced too close to the area of the macula on which the visual image is focused. Furthermore, laser treatment can only be applied only after sight-threatening changes have occurred. Finally, despite laser treatment, the disease and loss of vision may progress, and once vision is lost, it cannot be restored. No medical treatment is currently available for macular degeneration.

### SUMMARY OF THE INVENTION

We have now found in a cross-sectional survey of men and women that use of statins is associated with an 11-fold reduction in risk of macular degeneration. Statins are inhibitors of 3-hydroxy-3-methylglutaryl coenzyme A, i.e. HMG-CoA reductase inhibitors. Accordingly, we provide that age-related macular degeneration (AMD) is effectively treated by administration of HMG-CoA reductase inhibitors such as statins. Furthermore, administration of such HMG-CoA reductase inhibitors are effective in preventing the occurrence of age-related macular degeneration.

According to a first aspect of the present invention, we provide a method of treating or preventing macular degeneration in a patient, the method comprising administering an HMG-CoA reductase inhibitor to the patient.

There is provided, according to a second aspect of the present invention, a method of treating or preventing macular degeneration in a patient, the method comprising reducing the activity of an HMG-CoA reductase enzyme in a patient.

In a particular embodiment of the invention, treatment with HMG-CoA reductase inhibitors results in any one or more of the following: (i) reduced accumulation of basal linear deposit in Bruch's membrane; (ii) protection of the outer retina from oxidative damage; and (iii) inhibition of endothelial cell apoptosis.

We provide, according to a third aspect of the present invention, a method of using an HMG-CoA reductase inhibitor for the preparation of a pharmaceutical composition for the treatment or prevention of macular degeneration in a patient comprising: admixing said HMG-CoA reductase inhibitor with a pharmaceutically acceptable carrier so as to generate a pharmaceutical composition; and administering the pharmaceutical composition to the patient.

Preferably, the HMG-CoA reductase inhibitor comprises a statin. More preferably, the HMG-CoA reductase inhibitor comprises a statin selected from the group consisting of: fluvastatin (Lescol), cerivastatin (Baycol), atorvastatin (Lipitor), simvastatin (Zocor), pravastatin (Pravachol), lovastatin (Mevacor) and rosuvastatin (ZD 4522).

As a fourth aspect of the present invention, there is provided a method of treating or preventing macular degeneration in a patient, the method comprising one or more of the following: (a) lowering the level of LDL cholesterol in the patient; (b) increasing the level of HDL cholesterol in the patient; and (c) lowering the level of triglycerides in the patient.

Preferably, the level of LDL cholesterol is reduced by at least 20%, or in which the level of HDL cholesterol is increased by at least 5%, or in which the level of triglycerides is reduced by at least 10%.

Preferably, at least one of the following symptoms is reduced, eliminated, or prevented from developing or progressing in a patient: deterioration in sight, loss or reduction of visual acuity, loss of vision, distortion of vision, loss of central vision, increased macular pigment, presence of drusen, progression of small drisen to large drusen, progression of small drusen to confluent drusen, choroidal neovascularisation, progression of early disease to late, progression from dry to wet form or vice versa, geographic atrophy, RPE degeneration, RPE detachment, serous detachment of the sensory retina (SSR), retinal hard exudate, subretinal and/or sub-RPE haemorrhage, subretinal and/or sub-RPE fibrous tissue, retinal edema (thickening) and retinal haemorrhage.

More preferably, macular degeneration in a second eye is prevented from developing or progressing in a patient having macular degeneration in one eye.

In a preferred embodiment of the invention, the age-related macular degeneration comprises early macular degeneration. Alternatively, or in addition, the age-related macular degeneration comprises late macular degeneration. Preferably, progression of early to late age-related macular degeneration is prevented in the patient. More preferably, the treatments according to the invention result in a decrease of the incidence or progression of macular degeneration in the patient. Still more preferably, the methods of the invention prevent the incidence and/or progression of macular degeneration in a patient.

The present invention, in a sixth aspect, provides a method of predicting whether a patient is likely to develop macular degeneration, the method comprising determining whether the patient is suffering from, or has suffered from, stroke or coronary disease, in which if the patient has suffered or is suffering from stroke or coronary disease, then the patient is likely to develop macular degeneration.

Preferably, the patient is considered as suffering or having suffered from coronary disease if he is undergoing or has undergone one or more of the following: coronary bypass grafting; heart by-pass surgery; angioplasty; a heart attack; coronary artery disease and claudication.

Although the invention as set out above relates to macular degeneration, it should be noted that the methods, treatments and uses according to the invention may also be applied generally to treatment of any eye disease, including diseases of the retina, particularly degenerative diseases of the retina (retinal degenerative diseases or RDs). Examples of such degenerative diseases include retinitis pigmentosa, enhanced S-cone syndrome (ESCS), and Ushers syndrome. Other examples include myopic macular degneration, angioid streaks, presumed ocular histopasmosis syndrome, and other conditions with a propensity to the formatin of choroidal or sub-retinal neovascular membranes. Clinical definitions of such diseases and syndromes will be known to the skilled reader, with reference it necessary to any of the various textbooks on ophthalmology.

### DETAILED DESCRIPTION OF THE INVENTION

According to the invention, macular degeneration in a patient is treated or prevented by administration of HMG-CoA reductase inhibitors.

A patient is “treated” according to the invention if one or preferably more symptoms of macular degeneration as described below are eliminated or reduced in severity, or prevented from progressing or developing further. Preferably, at least one, more preferably, two, three, four, five or more of the symptoms as described below (in the sections headed “Nonexudative (dry) macular degeneration”, “Exudative (Wet) macular degeneration”, “Geographic Atrophy”, “Retinal Pigment Abnormalities”, “Detachment of the RPE”, “Choroidal Neovascularization (CNV, SRNVM)” and “Loss of Vision”) as being symptomatic of macular degeneration is reduced or eliminated, or prevented from progressing.

In another embodiment of the invention, treatment with the HMG-CoA reductase inhibitors prevents the incidence or progression of macular degeneration in an individual In this document, the term “incidence” should be taken to mean the appearance of a feature of macular degeneration at follow up when it was not present at the baseline examination. “Progression” in this document refers to an increase of two or more subfields in the area of involvement of a macular degeneration feature at follow up compared with baseline. Preferably, such features are chosen from the ones identified in the section “Features of Normal, Early and Late Macular Degeneration” below.

In a preferred embodiment of the invention, at least one of the following symptoms (where present) is reduced or eliminated, or its progression prevented: deterioration in sight, loss of vision, loss of central vision, presence of drusen, choroidal neovascularisation, progression of early disease to late, progression from dry to wet form or vice versa, geographic atrophy, RPE degeneration, RPE detachment, serous detachment of the sensory retina (SSR), retinal hard exudate, subretinal and/or sub-RPE haemorrhage, subretinal and/or sub-RPE fibrous tissue, retinal edema (thickening) and retinal haemorrhage.

Further symptoms which may be eliminated or reduced etc according to the invention include loss or reduction of visual acuity, distortion of vision, increased macular pigment, progression of small drusen to large drusen, progression of small drusen to confluent drusen. In a preferred embodiment of the invention, any one or more of the above symptoms is reduced or eliminated or its progression prevented in a second eye of a patient whose first eye is already affected to any extent (for example, by exhibiting any of the symptoms listed).

The reduction of a symptom as judged by a suitable grading system is preferably at least 10%, 15%, 20%, 30%, 40%, 50%, 60%, 70%, 80%, 90%, or more. It will be noted that some systems of grading of macular degeneration make use of discrete grades. Therefore, alternatively or in addition, administration of a HMG-CoA reductase inhibitor to a patient leads to an improvement of at least one grade, preferably two grades, more preferably three grades, four grades or more, in the relevant symptom as graded using a suitable system. For example, using WARMGS, a patient may exhibit grade 5 for maximum drusen size (i.e., drusen greater than or equal to circle C₂ (250 μm) in diameter). Treatment of the patient using HMG-CoA reductase inhibitor preferably leads to the patient exhibiting grade 4, preferably grade 3, more preferably grade 2, most preferably grade 1 or grade 0, in respect of drusen size. One or preferably more symptoms are reduced in grade by a treatment according to the invention.

A symptom is prevented from progressing where a patient is expected to exhibit a more severe form of the symptom (in the absence of treatment), but does not do so. The slowing down of progression of the disease, or a symptom of it, is preferably above a statistically significant level.

In a preferred embodiment, administration of HMG-CoA reductase inhibitors prevents a patient exhibiting drusen from developing macular degeneration. In another preferred embodiment, a patient with macular degeneration in one eye may be prevented from developing macular degeneration in the other eye. Furthermore, a patient with early stage macular degeneration may be prevented from developing late macular degeneration in another preferred embodiment. Other symptoms may be used to assess the effectiveness of treatment; for example, the central area of loss of vision where present may be prevented from developing further. An increase in drusen size, for example, as graded using the WARMGS, may be prevented. In a highly preferred embodiment of the invention, an improvement or remission in one or more signs or symptoms of macular degeneration is exhibited by a patient suffering from macular degeneration to whom a HMG-CoA reductase inhibitor is administered.

Each of the above progressions may be slowed down, by a treatment according to the invention. Preferably, the degree or stage of the disease in the patient undergoing treatment is compared with a patient from a similar background (e.g., a patient from a similar genetic, social, ethnic or age background). The degree or stage of the disease in the patient undergoing treatment may also be compared with a group of untreated patients. Methods of statistical analysis, as known in the art, and as set forth in the Examples, may be used to assess the effect of the treatment.

Preferably, a HMG-CoA reductase inhibitor is administered to a patient after (preferably as soon as or shortly after) signs of early disease are evident, in order to prevent (or prevent further) sight loss. Furthermore, individuals at high risk of progression (i.e. with signs of early disease) may be offered treatment to reduce their risk of losing central vision.

In another embodiment of the invention, prophylactic administration of a HMG-CoA reductase inhibitor to a patient prevents him from developing macular degeneration. Thus, the invention is suitable for prevention of the appearance of signs of early disease in previously unaffected individuals. A person is “prevented” from developing macular degeneration if he is considered statistically likely to develop the disease within a relevant timeframe, for example, 1 year, 2 years, 3 years, 4 years, 5 years, 10 years, 15 years, 20 years or more, but does not do so. This may be, and is preferably, established by comparison with a group of untreated people. A patient is considered “treated” according to the invention if the progression of macular degeneration disease is prevented or slowed, as compared to an untreated patient. For this purpose, a patient is preferably graded using a suitable system, for example, the WARMGS as described elsewhere in this document.

The HMG-CoA reductase inhibitor may be administered to the patient in any suitable form. The HMG-CoA reductase inhibitor may be administered in the form of a pharmaceutical composition, for example, as set out in detail below. Furthermore, foods and dietary supplements known to contain HMG-CoA reductase inhibitors as a natural ingredients may also be used. Examples of such foods include cholestin (Pharmanex) and Red Yeast Rice (rice fermented using the yeast Monascus purpureus). Red Yeast Rice contains several monacolins (including L, J and K) related to lovastatin and having HMG-CoA reductase inhibitor activity (Heber et al., 2001, Journal of Alternative and Complementary Medicine, Vol 7, Iss 2, pp 133-139A). Thus, any product derived from Monascus purpureus fermentation comprising statin activity may be used alone or in combination to treat or prevent macular degeneration.

Age-Related Macular Degeneration

As used in this document, the terms “macular degeneration”, “age-related macular degeneration” and “age-related maculopathy”, as well as the abbreviations “AMD”, “ARMD”, “ARM” are intended to be synonymous with each other.

Age-related macular degeneration is an acquired retinal disorder which is characterized by any of the following fundus changes: pigmentary atrophy and degeneration, drusen and lipofuscin deposits, and exudative elevation of the outer retinal complex in the macular area. Macular degeneration, which usually occurs in patients over age 55, results in progressive, sometimes significant, irreversible loss of central visual function from either fibrous scarring or diffuse, geographic atrophy of the macula. The definition can be expanded to include extrafoveal lesions that would have an impact on vision if superimposed on the foveal region (Bressler et al., 1988, Surv Ophthalmol 32:375-413.).

Nonexudative (Dry) Macular Degeneration

Nonexudative (dry or atrophic) macular degeneration accounts for 90 percent of all patients with macular degeneration in the United States. The disorder results from a gradual breakdown of the retinal pigment epithelium (RPE), the accumulation of drusen deposits, and loss of function of the overlying photoreceptors. Most patients with nonexudative macular degeneration experience gradual, progressive loss of central visual function. This loss of vision is more noticeable during near tasks, especially in the early stages of the disease. In an estimated 12-21 percent of patients, nonexudative macular degeneration progresses to cause vision levels of 20/200 or worse (Murphy, Ophthalmology 1986; 93:969-71; Hyman, Am J Epidemiol 1983; 118:213-27;.Smiddy and Fine, Ophthalmology 1984; 91:271-7). Both choroidal neovascularization (choroidal neovascularization) and subretinal or sub-retinal pigment epithelium exudation are conspicuously absent in this category of macular degeneration.

Exudative (Wet) Macular Degeneration

Although exudative (wet) macular degeneration accounts for only 10 percent of patients with macular degeneration, 90 percent of the macular degeneration patients with significant vision loss have this form of the disease (Hyman et al., Am J Epidemiol 1983; 118:213-27; Ferris, Am J Epidemiol 1983; 118:132-51; Ferris et al., Arch Ophthalmol 1984; 102:1640-2). Exudative macular degeneration is characterised by the development of neovascularization in the choroid, leading to serous or hemorrhagic leakage and subsequent elevation of the retinal pigment epithelium or neurosensory retina. Patients with exudative macular degeneration tend to notice a more profound and rapid decrease in central visual function. Serous or hemorrhagic leakage from the new choroidal vessels causes dysmorphopsia, scotoma, and blurred vision (Fine et al., Arch Ophthalmol 1986; 104:513-4).

Each of the two forms of macular degeneration has its own features. In most patients nonexudative macular degeneration will not progress to severe vision loss. Those patients in whom macular degeneration progresses to the exudative form are at greatest risk for severe visual impairment. Patients who have exudative maculopathy with drusen in the fellow eye are at significant risk of developing choroidal neovascularization (Strahlman et al., Arch Ophthalmol 1983; 101:1191-3).

Geographic Atrophy

Geographic atrophy is a clinical manifestation of progressive atrophy of the retinal pigment epithelium in conjunction with drusen formation. In most cases the natural history of the disease reflects retinal atrophy following the fading of drusen (Wing et al., Invest Ophthalmol Vis Sci 1978; 17:601-7). From one to several well-demarcated areas of retinal pigment epithelium atrophy are accompanied by overlying photoreceptor damage. Single or multiple areas of well-defined atrophy spread throughout the foveal and parafoveal area, producing a gradual decrease in vision. The process is diffuse, often bilateral and symmetrical, and it can lead to vision loss even in the absence of choroidal neovascularization. Choroidal neovascularization can develop as a separate entity in the presence of soft, confluent drusen. Geographic atrophy can also follow the collapse of a retinal pigment epithelial detachment though it is uncommon, occurring in less than 2 percent of serous retinal pigment epithelium detachment cases within 2 years of diagnosis (Merideth et al., Am J Ophthalmol 1979; 88:643-51; Bird and Marshall, Trans Ophthalmol Soc UK 1986 105:674-82; Elman et al., Ophthalmology 1986; 93:224-30; Poliner et al., Ophthalmology 1986; 93:543-51). Sometimes geographic atrophy occurs after an retinal pigment epithelium tear or rip and can be associated with ill-defined or occult choroidal neovascular membranes (CNVM).

Retinal Pigment Abnormalities

Retinal pigment abnormalities are considered the earliest retinal manifestations of macular degeneration and consist of increased retinal pigmentary degeneration and atrophy in the plane of the retinal pigment epithelium. A grayish-yellow or pinkish-yellow area in the macula is often surrounded by a halo of gray or black pigment clumps in or beneath the retina. Increased lipofuscin in the retinal pigment epithelium and the accumulation of debris on and within Bruch's membrane result in loss of photoreceptor function.

Drusen

Drusen are yellow to yellowish-white nodular deposits found in the deeper layers of the retina and comprise hyaline deposits or colloid bodies of Bruch's lamina of the choroid, very commonly present but not always affecting vision. Drusen are most often seen as a consequence of aging, but are found histologically in younger persons. Drusen are frequently associated with the subsequent development of macular degeneration. Clinical features associated with an increased risk of subsequent visual loss are large soft drusen and/or confluent drusen, in addition to focal hyperpigmentation or the RPE, particularly if one eye has already developed visual loss from AMD.

A detailed review of the pathogenesis of drusen in age-related macular degeneration is provided by Abdelsalam et al., 1999, Survey of Ophthalmology, 44(1), 1-29.

Drusen may vary in number, size, shape, degree of elevation and extent of associated changes in the RPE. Usually occurring in clusters, drusen can be found anywhere in the posterior pole. In some patients, drusen may be confined to the region of the fovea, whereas in others the deposits encircle the fovea but spare the fovea itself. Drusen can occur outside the vascular arcades and are often found on the nasal side of the optic disc. When found in the equatorial region, they are often accompanied by a reticular pattern of pigmented lines. Soft drusen are confluent lesions greater than 63 microns in diameter, that merge and touch and lack sharp borders (Gass, Arch Ophthalmol 1973; 90:206-17).The terms “semisolid” or “mixed” drusen have been used to dcscribe the clinical picture comprising both types of lesions.

Several types of drusen have been described. The lesions are categorized by size, confluence, uniformity, and sharpness of borders (Strahlman et al., Arch Ophthalmol 1983; 101:1191-3; Gass, Arch Ophthalmol 1973; 90:206-17.). Some form of drusen are found in the macular area in 50-95 percent of persons over age 70. Among persons with drusen, 10-15 percent may eventually develop exudative manifestations of macular degeneration (Bressler et al., Surv Ophthalmol 1988; 32:375-413; Kahn et al. Am J Epidemiol 1977; 106:33-41; Klein and Klein, Arch Ophthalmol 1982; 100:571-3.).

Hard Drusen

Hard drusen are small, round, discrete, yellow-white spots which are associated with focal dysfunction of the RPE. In the majority of patients they are innocuous.

Soft Drusen

Soft drusen are larger than hard drusen and have indistinct edges. With time they may slowly enlare and coalesce. Confluent drusen are associated with diffuse dysfunction of the RPE and an increased risk of subsequent CNV.

Basal Laminar Drusen

Basal laminar drusen are innumerable, small, uniform, round, subretinal nodules. They occur in patients who are younger than those seen with hard or soft drusen and may be associated with pseudo vitelliform detachment of the sensory retina, or rarely with CNV.

Calcified Drusen

Calcified drusen have a glistening appearance secondary to dystropic calcification.

Drusen may be graded by any suitable grading system, for example, as used in the WARMGS. This particular grading system for drusen is described in detail at pages 1129 to 1131 of the paper (Klein et al, 1991, Ophthalmology 98, 1128-1134) and categorises drusen according to maximum size, predominant type, area, and degree of confluence (touching or merging of two or more drusen). In this system, drusen size and type are graded with the aid of standard circles with diameters corresponding to 63 μm, 125 μm and 250 μm.

Detachment of the RPE

Detachment of the retinal pigment epithelium may be another symptom exhibited by patients with macular degeneration. Such detachment is manifested as a sharply circumscribed, dome-shaped elevation at the posterior pole of varying size. Fluorescein angiography shows free fluorescein pools in the sub-RPE space giving rise to an area of hyperfluorescence, while ICG angiography shows a well defined area of hypofluorescence. Patients exhibiting RPE detachment may subsequently course exhbit any of the following; spontaneous resolution, geographic atrophy, detachment of the sensory retina, development of occult choroidal neovascularisation, tear of the RPE with development of CNV.

Choroidal Neovascularization (CNV, SRNVM)

Choroidal neovascularization is the proliferation of fragile, newly formed blood vessels originating in the choroidal space and penetrating through to the outer retinal complex into the subretinal and retinal tissue. Serous or haemorrhagic leakage from these vessels results in a neurosensory or retinal pigment epithelial detachment.

Diffuse thickening of Bruch's membrane, in conjunction with soft, confluent drusen and pigment abnormalities, predisposes the patient to the development of a choroidal neovascular membrane. The new vessels of the CNVM form an organized yet fragile vascular system. As the system matures, the delicate neovascular twigs leak fluid (protein and lipids) into the subretinal, intraretinal, or sub-retinal pigment epithelium space. Depending on numerous factors, the hemorrhage at the site of the membrane or in the subretinal space may extend into the vitreous.

In the case of sub-retinal pigment epithelium hemorrhage, direct examination of the macula reveals a discrete round to oval elevated lesion. Often surrounded by a subretinal hemorrhage, the lesion appears greenish-gray or dirty brown. A halo of pigment may also surround the lesion. Other findings include subretinal lipid or blood, associated with an overlying thickened, detached neurosensory retina. This insidious lesion, which tends to be rapid growing and recurrent, heralds the onset of late macular degeneration and exudative maculopathy.

When found with macular degeneration, the lesions tend to be larger, probably because they are part of a more diffuse retinal disease process. These lesions are often found under the center of the FAZ which explains their more devastating effect on central visual function (Green and Wilson, Ophthalmology 1986; 93:1169-76; Bressler et al., Arch Ophthalmol 1987; 105:209-13). Choroidal neovascularization, when associated with macular degeneration, seems to induce more damage to the retinal tissue than that caused by other choroidal diseases, such as presumed ocular histoplasmosis syndrome, angioid streaks, or idiopathic causes of choroidal neovascularization (Bressler et al.,. Surv Ophthalmol 1988; 32:375-413).

Choroidal neovascularization has been divided into two categories on the basis of clinical and fundus fluorescein angiographic appearance. Well-defined CNVs arc delineated in their entirety during the carly stages of the fluorescein angiogram. A further designation refers to those lesions which may not be entirely visible in the early stages of the fluorescein transit; however, the entire lesion is visible in the later stages following leakage. Classic, well-defined choroidal neovascularization, which fluoresces early in the angiogram, has a lacy appearance, with the cartwheel appearance of the new vessels sometimes apparent. The neurosensory retina has a hyperfluorescent pool of fluid in the late stages of the angiogram. Poorly defined membranes are difficult to identify due to the presence of subretinal blood, turbid fluid, or pigment. Half of the membranes are ill-defined and located within 200 microns of the center of the FAZ (Roy and Kuehl, Ann Ophthalmol 1989; 21:429-31).

A number of retinal pigment epithelial lesions occur in conjunction with choroidal neovascularization or occur as a consequence of the new vessel formation. A serous retinal pigment epithelial detachment (PED) resulting from leakage of new choroidal vessels is characterized ophthalmoscopically as a well-demarcated, dome-shaped, elevated yellowish-orange lesion. The overlying neurosensory retina may be detached, and the separation often exceeds the boundaries of the PED, providing further evidence of choroidal neovascularization. Hemorrhagic retinal pigment epithelium detachment appears as a red or dark green elevated lesion. It may affect vision if located under the fovea. A hemorrhagic PED can mimic a choroidal melanoma and indicates the possible presence of a choroidal tumor with subretinal hemorrhage. Chorioretinal folds adjacent to the PED are sometimes observed. Long-standing detachments may lead to retinal pigment epithelium atrophy or hypopigmentation in the area of the lesion. Patients over age 55 with retinal pigment epithelium detachment and accompanying drusen are at significant risk of developing severe vision loss from choroidal neovascularization (Merideth et al., Am J Ophthalmol 1979; 88:643-51; Elman et al., Ophthalmology 1986; 93:224-30; Poliner et al., Ophthalmology 1986; 93:543-51).

Retinal pigment epithelial tears or rips occur spontaneously or follow laser photocoagulation of choroidal neovascularization. These retinal pigment epithelium tears are a more consistent finding with occult or ill-defined choroidal neovascularization, usually occurring adjacent to the site of new choroidal vessels. Occasionally, retinal pigment epithelium tears are detected with disciform scars and usually result in severe vision loss (Gass, Br J Ophthalmol 1984; 68:513-9).

Vitreous hemorrhage can occur with exudative macular degeneration. When the patient reports sudden vision loss, it is sometimes the result of a breakthrough haemorrhage. Although the vitreous haemorrhage clears in 75 percent of patients, ultrasonography and stereoscopic fundus examination are indicated to rule out causes other than macular degeneration (Tani et al., Am J Ophthalmol 1980; 90:525-33).

A fibrovascular disciform scar represents the final stage of untreated choroidal neovascularization. A yellowish-white to brown or black lesion is observed in the macula. Subretinal fluid or fresh hemorrhage frequently appears at the edges of the scar. Hypertrophic retinal pigment epithelium, chorioretinal folds, and anastomosis of the retinal and choroidal circulations also have been observed in advanced choroidal neovascularization.

Loss of Vision

Vision loss from macular degeneration can develop in one of several different ways. The most frequent cause of significant, acute vision loss is exudative maculopathy, where serous or haemorrhagic detachment of the neurosensory retina results from leakage of new vessels in the choroid. Another cause for vision loss is geographic atrophy, characterised by degeneration and loss of retinal pigment epithelium. This process damages the overlying photoreceptors and produces a chronic decrease in visual function.

Symptoms of dysmorphopsia, blurred vision (especially at near) or central scotoma that accompany drusen or retinal pigment epithelium atrophy should arouse suspicion of choroidal neovascularization.

Stages of Macular Degeneration

Numerous systems have been proposed for classifying the various stages of macular degeneration. Most of these systems rely on the ophthalmoscopic appearance of the individual macular lesions, the extent of involvement of the macula, and the patient's visual acuity (Klein et al., Ophthalmology 1991; 98:1128-34).

An example of such a classification is shown in Table 1 below, which includes a classification of the disease according to “early” or “late” forms. Other classifications are disclosed in detail below.

TABLE 1
Example Classification of Stages of macular degeneration

| | |
|---|---|
| 1. | Pigmentary abnormalities: |
| | retinal pigment epithelium degeneration |
| | Increased retinal pigment in the macular area |
| 2. | Retinal pigment epithelial degeneration and increased retinal |
| | pigment: |
| | Granules or clumps of gray or black pigment in or beneath the |
| | retina |
| | Grayish-yellow or pinkish-yellow areas of varying density and |
| | configuration in the plane of the retinal pigment epithelium |
| 3. | Early macular degeneration: |
| | Soft, indistinct, or reticular drusen |
| | Any type of drusen (except hard, indistinct) with retinal pigment |
| | epithelium degeneration in the absence of signs of late macular |
| | degeneration |
| 4. | Late macular degeneration: |
| | retinal pigment epithelium or serous detachment of the |
| | sensory retina |
| | Subretinal or pigment epithelium hemorrhage |

Pure geographic atrophy may also occur, characterised by geographic atrophy in the absence of exudative macular degeneration, together with loss of retinal pigment epithelium and increased visualization of the underlying choroid.

Features of Normal, Early and Late Macular Degeneration

The early stage of macular degeneration is recognizable by macular changes consisting of retinal pigment epithelium abnormalities and typical, soft, and indistinct drusen. Patients who have fundus findings in this category can be considered at risk for severe vision loss especially if choroidal neovascularization develops later. Late or advanced stages of macular degeneration arc characterized by the presence of choroidal neovascularization with exudation, consisting of blood, lipid, or serosanguinous material, that produces rectinal pigment epithelium or neurosensory detachment. The final stage of the exudative form of macular degeneration consists of a disciform scar in the foveal area with severe loss of central visual function.

The following paragraphs set out in detail definitions of normal, early and late macular degeneration which may be employed in a highly preferred embodiment of the invention.

According to this embodiment, an individual is defined as suffering from early macular degeneration according to WARMGS if one or more of the following characteristics identified from fundus photographs are found in either or both eyes within a standard 3000 micron grid centered on the centre of the fovea: (a) soft indistinct or reticular drusen in either eye; (b) hard or soft drusen with increased or decreased retinal pigment in either eye; or (c) absence of atrophic (dry) or exudative (wet) macular degeneration in both eyes.

Similarly, according to this embodiment, an individual is defined as suffering from late macular degeneration if, as judged by WARMGS, one or more of the following characteristics identified from fundus photographs are found in either or both eyes within a standard 3000 micron grid centered on the centre of the fovea: (a) retinal pigment epithelial detachment and serous detachment of the sensory retina (RPE/SSR detachment, preferably as characterised by sharply defined, solid-looking dome-shaped elevations of the RPE, which are round, oval or kidney-shaped; (b) hard exudate (retinal and subretinal), preferably as characterised by small white or yellowish white deposits with sharp margins which may be arranged as individual dots, confluent patches, or in rings partially surrounding zones of retinal oedema; (c) subretinal and/or sub RPE haemorrhage; (d) Subretinal fibrous scar (or fibrin), preferably as characterised by the presence of sheets or mounds of white material under the retina; (e) geographic atrophy, preferably as characterised by sharply defined area of drop-out or depigmentation of RPE exposing the underlying choroid which must be at least as large as circle 1-1 of the WARMGS.

Finally, according to this embodiment, an individual is considered to be “normal” with respect to macular degeneration if he or she exhibits none of the signs of macular degeneration according to WARMGS. In other words, according to this embodiment, a person is “normal” if the following characteristics identified from fundus photographs are found in either or both eyes within a standard 3000 micron grid centred on the centre of the fovea: (a) no evidence of early (see below) or late (see below) macular degeneration in either eye; or if the largest drusen size is 250 microns or less, the most severe type is “soft distinct” and there is no evidence of increased or decreased retinal pigment in either eye.

According to a highly preferred embodiment of the invention, administration of a statin or HMG-CoA reductase inhibitor to a patient suffering from macular degeneration results in prevention of early or late (preferably early) macular degeneration developing in a normal patient, or prevention of late macular degeneration developing in a patient suffering from early macular degeneration, according to the above definitions. According to this embodiment, administration of a statin to a patient who is suffering from early or late macular degeneration in one eye prevents early or late macular degeneration developing in the other eye of the patient.

Grading Systems

The progression of macular degeneration disease is assessed by various ways as known in the art; each of these methods may therefore also be used to assay the effectiveness of the treatments and methods described here.

Macular degeneration may be assessed by a simple visual acuity test, for example, one as commonly applied by optometrists. In such a test, the subject may be made to read and identify a series of symbols or letters of varying sizes (or at varying distances) on an eye chart. Such an eye chart test may be combined or supplemented with self-reporting.

More preferred methods of assessment include visual examination of the retinal area by a specialist, for example, a trained ophthalmologist. Such ophthalmoscopy may be direct or indirect, and may include scanning laser ophthalmoscopy. Furthermore, the presence of drusen (an early indicator of macular degeneration) may be observed and quantified. Wet macular degeneration may also be diagnosed and the degree of this disease assessed by asking the subject to view an Amsler grid. The Amsler grid is a checkerboard pattern, the straight lines of which are perceived to be wavy in patients with wet macular degeneration.

Furthermore, the status of blood vessels in the retina, for example, whether they are leaking, may be assessed by various means. Such means include fluorescein or indocyanine green angiography, or other dye injection to aid visualisation. Fundus photography, including stereoscopic fundus photography, may also be used.

Any of the above assessment methods may preferably be quantified by grading, for example by an ophthalmologist or other experienced observer. Such grading may be, and is preferably, used to assess the efficacy of treatment. Visible macular changes which may be observed and graded in a grading system may include drusen, atrophy, increased/decreased pigmentation and scarring. These changes may be observed and recorded by for example an experienced observer (e.g. ophthalmologist) using an ophthalmoscope.

A particularly preferred test for macular degeneration is the WARMGS, described in detail below, which provides a quantitative assessment of the degree or severity of the disease. However, other grading systems for macular degeneration may also be used, for example, the Alabama Age-Related Macular Degeneration Grading System, as described in Curcio et al., Invest Ophthalmol Vis Sci 1998; 39:1085-96. Furthermore, an International Classification and grading system for age-related maculopathy and age-related macular degeneration has been described (The International ARM Epidemiological Study Group, 1995, Survey of Ophthalmology 39, 367-374). This classification is derived from, and uses, similar protocols to the WARMGS and may also be used to grade AMD.

Wisconsin Age Related Maculopathy Grading System (WARMGS)

The best data on incidence and progression of early and late macular degeneration come from a follow-up study of the residents of Beaver Dam Wis. In this study, the investigators put forward a technical definition of early macular degeneration based on the size and appearance of drusen and the presence of pigment change. Though other grading systems exist (e.g. Moorfields system) the task of surveying large numbers of subjects over a lengthy time period to determine the “high risk” characteristics of early disease make it unlikely that the WARMGS will be superseded.

The Wisconsin Age Related Maculopathy Grading System (WARMGS) provides a robust and reproducible grading system which is essential for studying the natural history of macular degeneration and how it might be modified by drugs or other influences. Grading of stereoscopic fundus photographs using WARMGS, for example as performed in the study set out in the Examples, is regarded as the gold-standard for epidemiological studies of the disease. WARMGS is therefore a preferred method for assessing the progression and severity of macular degeneration according to the invention.

The Wisconsin Age Related Maculopathy Grading System (WARMGS) is described in detail in Klein et al, 1991, “The Wisconsin Age-related Maculopathy Grading System” Ophthalmology 98, 1128-1134 accession number PB91184267. Preferably, a subject is graded by comparison to photographic standards described in the above reference; the relevant photographic standards are produced by the Department of Ophthalmology, University of Wisconsin Medical School, 610 N. Walnut Street, Madison, Wis. and are available from the US Department of Commerce National Technical Information Service, Springfield, Va. 22161.

HMC-CoA Reductase Inhibitors/Statins

The term “statin” is used in the art to denote a class of drugs which have inhibitory activity against 3-hydroxy-3-methyl-glutaryl-coenzyme A (HMG-CoA) reductase. A statin is therefore an agent which inhibits an activity of HMG-CoA reductase, preferably conversion of HMG-CoA to mevalonate. Preferably, the agent inhibits the activity of human HMG-CoA reductase, preferably a human HMG-CoA reductase having accession number M11058. The structure of human HMG-CoA reductase is described in detail in Luskey and Stevens (1985), J. Biol. Chem. 260 (18), 10271-10277, which also discloses a cDNA sequence for HMG-CoA reductase, accession number M11058.

Accordingly, although the terms “statin” and “HMG-CoA reductase inhibitor” are used interchangeably in this document, the invention is primarily concerned with the class of drugs which exhibit HMG-CoA reductase inhibitory activity. Thus, the invention includes such drugs and compounds having this activity, whether currently known or to be discovered, even where such a drug may not be commonly known or referred to as a “statin”. These terms should also be taken to include derivatives of the compounds concerned, for example, salts, including metal salts such as sodium salts.

HMG-CoA reductase is an important enzyme in cholesterol biosynthesis. As a first step in cholesterol biosynthesis acetyl-CoAs are converted to 3-hydroxy-3-methylglutaryl-CoA (HMG-CoA). HMG-CoA is then converted to mevalonate by HMG-CoA reductase, using NADPH as a co-factor. The reduction of HMG-CoA to mevalonate is a rate-limiting step in cholesterol biosynthesis. Mevalonate is converted to the isoprene based molecule, isopentenyl pyrophosphate (IPP), with the concomitant loss of CO₂, IPP is converted to squalene, and finally, squalene is converted to cholesterol. In general, statins act by competitively inhibiting 3-hydroxyl-3-methylglutaryl-coenzyme A (HMG-CoA) reductase. The reaction catalyzed by HMG-CoA reductase is the rate limiting step of cholesterol biosynthesis, and this enzyme is subject to complex regulatory controls.

As a result of inhibition of HMG-CoA to mevalonate conversion, a compensatory increase in the expression of LDL receptors on hepatocyte membranes and a stimulation of LDL catabolism occurs. Statin administration therefore results in an increase in HDL while total, LDL and VLDL cholesterols, apolipoprotein B, and plasma triglycerides are decreased. Physiologically, therefore, HMG-CoA reductase inhibitors are capable of lowering low-density lipoprotein (LDL) and total cholesterol levels. In addition, HMG-CoA reductase inhibitors are capable of lowering triglyceride levels in patients with isolated hypertriglyceridaemia.

The observed effects of HMG-CoA reductase inhibitors in treating macular degeneration may therefore be due to their effects on cholesterol metabolism and balance. We therefore propose that altering cholesterol levels and in particular the balance between HDL and LDL may be used to treat or prevent age-related macular degeneration. Accordingly, the invention provides for a method of treating or preventing macular degeneration in a patient, the method comprising one or more of the following: (a) lowering the level of LDL cholesterol in the patient; (b) increasing the level of HDL cholesterol in the patient; and (c) lowering the level of triglycerides in the patient. Preferably, the level of LDL cholesterol is reduced by at least 20%, or in which the level of HDL cholesterol is increased by at least 5%, or in which the level of triglycerides is reduced by at least 10%.

Other non-lipid-lowering effects are generally exhibited by HMG-CoA reductase inhibitors, including improvement in endothelial function, antiproliferative actions on smooth muscle cells, upregulation of endothelial nitric oxide synthase and reduction in platelet aggregation. Anti-inflammatory effects and reduction of plasma glucose levels may also be observed in certain HMG-CoA reductase inhibitors. The disease modifying properties of HMG-CoA reductase inhibitors have been used to treat stroke and dementia (Jick et al., 2000, Lancet 356, 1627-1631).

The discovery that HMG-CoA reductase inhibitors are useful for treating age-related macular degeneration, and their known role in cholesterol metabolism and in treatment of stroke and other coronary diseases suggests that the same mechanism may be responsible for both types of diseases. Diagnosis or observation of one disease in a patient may therefore be used as an aid to predict the likelihood of the other developing. Accordingly, we provide a method of predicting whether a patient is likely to develop macular degeneration, the method comprising determining whether the patient is suffering from, or has suffered from, stroke or coronary disease, in which if the patient has suffered or is suffering from stroke or coronary disease, then the patient is likely to develop macular degeneration. The reverse prediction is also provided. Preferably, the patient is considered as suffering or having suffered from coronary disease if he is undergoing or has undergone one or more of the following: coronary bypass grafting; heart by-pass surgery; angioplasty; a heart attack; coronary artery disease and claudication.

An HMG-CoA reductase inhibitor suitable for use in the present invention preferably inhibits the activity of HMG-CoA reductase by at least 5%, 10%, 20%, 30%, 40%, 50%, 60%, 70%, 80%, 90%, 95% or more. A protocol for assaying inhibitory activity is set out in detail below.

Preferably, the HMG-CoA reductase inhibitor is selected from the group consisting of: Atorvastatin, Cerivastatin, Compactin, Crilvastatin, Dalvastatin, Dihydroeptastatin, Dihydromevinolin, Eptastatin (CS-514), Fluvastatin, Lovastatin, Mevacor, Mevalotin, Mevastatin, Mevinolin, ML-236B, Monacolin J, Monacolin K, Monacolin L, Monacolin X, Pravastatin, RG 12561, Simvastatin, Lescol, Lipitor, Lipobay, Pravacol and Zocor.

In a highly preferred embodiment of the invention, the statin is selected from the group consisting of: fluvastatin (Lescol), cerivastatin (Baycol), atorvastatin (Lipitor), simvastatin (Zocor), pravastatin (Pravachol), lovastatin (Mevacor) and rosuvastatin (ZD 4522). A further example of a statin which may be used is NK-104. NK-104 is (+)-monocalcium bis((3R,5S,6S)-7-[2-cyclopropyl-4-(4-fluorophenyl)-3-quinolyl]-3,5-dihydroxy-6-heptenoate, CAS 147526-32-7, and the cholesterol-lowering effect of this compound has been described in detail in Aoki et al., 2001, Arzneimittelforschung;51(3):197-203.

Each of the above compounds is known in the art. For example, Simvastatin is described in detail in Hess et al., 2001, Expert Opin Pharmacother;2(1):153-63, while atorvastatin (CAS 134523-00-5) is described in detail in Wierzbicki et al., 2001, Expert Opin Pharmacother;2(5):819-30. McTaggart et al., 2001, Am J Cardiol, 87(5 Suppl 1):28-32 describe the properties of rosuvastatin, which was previously known as ZD4522.

Other HMG-CoA reductase inhibitors, for example as disclosed in U.S. Pat. No. 6,218,403, U.S. Pat. No. RE 36,481 and U.S. Pat. No. RE36,520 may also be employed. Furthermore, U.S. Pat. Nos. 5,877,208, 5,792,461 and 5,763,414 disclose the use of naringin and naringenin, citrus peel extract and hesperidin and hesperetin respectively as HMG-CoA reductase inhibitors.

As noted above, HMG-CoA reductase inhibitors for use in the invention may comprise compounds presently known to inhibit HMG-CoA reductase activity. Alternatively, they may comprise known or unknown compounds which are not previously known to inhibit HMG-CoA reductase. Such compounds may comprise candidate compounds, which when tested are found to comprise HMG-CoA reductase inhibitor activity. Candidate compounds suspected of having HMG-CoA reductase inhibitor activity may be tested by means of various assays of HMG-CoA reductase activity known in the art, for example as set out below. Such compounds may be tested in the form of a library, for example a combinatorial library, as known in the art.

The HMG-CoA reductase inhibition activity of a known HMG-CoA reductase inhibitor or a candidate compound may be measured in the in vitro protocol published in J. Med. Chem., 28, p. 47-358 (1985) and described below:

Isolation of HMG-CoA Reductase

Male Holtzman Sprague-Dawley rats (225-250 g) are kept on reversed lighting and fed Purina rat chow containing 3% cholestyramine for 7 days preceding their sacrifice by CO₂ asphyxiation. Livers are removed 6 hours into the dark cycle and used immediately to prepare microsomes. HMG-CoA reductase is solubilized from the freshly prepared microsomes by the method of Heller and Shrewsbury [J. Biol. Chem., 1976, 251, 3815]and purified through the second ammonium sulfate precipitation step as described by Kleinsek et al. [Proc. Natl. Acad. Sci. USA, 1977, 74, 1431]. The enzyme preparation is tested for HMG-CoA reductase potency and diluted with 100 mM phosphate buffer (pH 7.2) so that 100 μl of the enzyme solution, when added to the assay control, gives a value of 50,000-60,000 dpm. The enzyme preparation is stored at −80 degrees C.

HMG-CoA Reductase Inhibition Assay

The assay is essentially the procedure of Shefer et al [J. Lipid Res., 1972, 13, 402]. The complete assay medium contains the following in a total volume of 0.8 ml: phosphate buffer, pH 7.2, 100 mM; MgCl₂, 3 mM; NADP, 3 mM; glucose-6-phosphate, 10 mM; glucose-6-phosphate dehydro-genase, 3 enzyme units; reduced glutathione 50 mM; HMG-CoA (glutaryl-3-14 C, New England Nuclear), 0.2 mM (0.1 μCi); and partially purified enzyme stock solution, 100 μL.

Test compounds or compactin, after first being converted to the sodium salt of their dihydroxy acid form in situ by addition of 1N NaOH (1 equivalent), are added to the assay system in 10-μL volumes at multiconcentration levels. After a 40-minute incubation at 37 degrees C. with shaking and exposure to air, the reaction is stopped by the addition of 0.4 mL of 8 N HCl. After an additional 30-minute incubation period at 37 degrees C. to ensure the complete lactonization of mevalonic acid to mevalonolactone, 0.2 ml of the mixture is added to an 0.5×5.0 cm column containing 100-200 mesh Bio-Rex 5, chloride form (Bio-Rad), wetted with distilled water, as described by Alberts et al. [Proc. Natl. Acad. Sci. U.S.A., 1980, 77, 3967]. The unreacted [¹⁴C]HMG-CoA is absorbed on the resin and the [¹⁴C]mevalonolactone is eluted with distilled water (2×1 ml) directly into 7-ml scintillation vials. Five milliliters of Aquasol-2 (New England Nuclear) is added to each vial, and radioactivity measured in a Packard Tri Carb Prias scintillation counter. IC₅₀ values are determined by plotting percentage inhibition against test compound concentration and fitting a straight line to the resulting data by using the least-squares method. For estimation of relative inhibitory potencies, compactin is assigned a value of 100 and the IC₅₀ value of the test compound is compared with that of compactin determined simultaneously.

Pharmaceutical Compositions and Dosage Forms

According to the invention, a HMG-CoA reductase inhibitor is administered to a patient suffering, or likely to be suffering, from age-related macular degeneration. Advantageously the HMG-CoA reductase inhibitor is present in an amount to treat or prevent macular degeneration in a subject in need thereof. Preferably, the HMG-CoA reductase inhibitor is provided in the fonn of a pharmaceutical composition which contains the drug in a dosage unit in an amount from about 0.01-200 mg, preferably 1-100 mg, together with a pharmaceutically acceptable carrier or diluent.

The HMG-CoA reductase inhibitors may be administered to the patient in any suitable form, for example, in a form as presently known to be suitable for treatment of heart disease, high cholesterol levels, etc. The pharmaceutical carrier may be, for example, either a solid or a liquid. The administration may be parenterally, rectally, topically, transdermally or orally, the latter being the preferred route of administration. The pharmaceutical forms are, for example, syrups, suspensions or emulsions, tablets, capsules and lozenges.

A liquid formulation will generally consist of a suspension or solution of the compound or pharmaceutically acceptable salt in a suitable liquid carrier(s) for example, ethanol, glycerine, non-aqueous solvent, for example, polyethylene glycol, oils, or water with a suspending agent, preservative, flavouring or colouring agent.

The present invention also provides for a controlled release formulation to be administered to a mammal comprising a mixture of one or more HMG-CoA reductase inhibitors, or pharmaceutically acceptable salts thereof, a water-channelling agent and a wetting agent. The mixture is in the form of a non-compressed pellet, having an enteric coat or a sustained release coat permeable to gastrointestinal juices. These slow release pharmaceutical compositions are prepared, for example, as described in U.S. Pat. No. 4,524,060, issued Jun. 18, 1985. Other controlled release formulations arc described in U.S. Pat. No. 4,880,830, issued Nov. 14, 1989 and U.S. Pat. No. 5,068,112, issued Nov. 26, 1991.

HMG-CoA reductase inhibitors may be administered singly, or in combination with other drugs, including other cholesterol reducing drugs and/or other HMG-CoA reductase inhibitors. Cholesterol reducing drugs are known in the art. Where more than one drug is administered, this may be done sequentially or simultaneously. The drugs may be pre-mixed and administered together, or administered in separate aliquots substantially simultaneously.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

A composition for parenteral administration which can be formulated as a solution or a suspension will generally consist of a solution or suspension of the active ingredient in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

A typical suppository composition comprises a compound of the instant invention or a pharmaceutically acceptable salt thereof which is active when administered in this way, with a binding and/or lubricating agent such as polymeric glycols, gelatins or coca butter or other low melting vegetable or synthetic waxes or fats.

A typical transdermal formulation comprises a conventional aqueous or non-aqueous vehicle, for example, a cream, ointment lotion or paste or in the form of a medicated plaster, patch or membrane.

For topical administration, the pharmaceutical compositions adapted include solutions, suspensions, ointments, and solid inserts. Typical pharmaceutically acceptable carriers are, for example. water, mixtures of water and water-miscible solvents such as lower alkanols or vegetable oils, and water soluble ophthalmologically acceptable non-toxic polymers, for example, cellulose derivatives such as methyl cellulose. The pharmaceutical preparation may also contain non-toxic auxiliary substances such as emulsifying, preserving, wetting, and bodying agents, as for example, polyethylene glycols; antibacterial components such as quaternary ammonium compounds; buffering ingredients such as alkali metal chloride; antioxidants such as sodium metabisulfite; and other conventional ingredients such as sorbitan monolaurate.

Furthermore, stabilized pharmaceutical compositions comprising an HMG-CoA reductase inhibitor compound, for example, as described in U.S. Pat. No. 5,356,896 may also be employed.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral, parenteral, rectal, transdernal, or topical products.

A suitable dosage may be determined empirically, for example using as starting point a dosage suitable for treatment of heart disease or high cholesterol. For atorvastatin, for example, the dosage is suitably about 10 to 80 milligrams (mg) once a day. For cerivastatin, the dosage is suitably about 0.4 milligrams (mg) once a day in the evening. For fluvastatin, the dosage is suitably about 20 to 40 milligrams (mg) once a day in the evening. For lovastatin, the dosage is suitably about 20 to 80 milligrams (mg) a day. For pravastatin, the dosage is suitably about 10 to 40 mg once a day at bedtime. For simvastatin: 5 to 40 mg once a day in the evening.

The above dosages may be taken as a single dose or divided into smaller doses. Ingestion with meals may be necessary depending on the nature of the drug. Dosages for children will generally be smaller than those for adults, and a physician may need to be consulted in order to determine the dosage.

### EXAMPLES

### Example 1.

Summary of Study

As part of a study of age-related eye diseases, we recorded a drug history, and report here an association between use of statins and reduced risk of age-related macular degeneration.

741 men and women aged 66 to 75 who had been traced by the Office for National Statistics (United Kingdom) using information from their birth records at the Jessop Hospital for Women, Sheffield, United Kingdom were approached. 412 (56%) agreed to be interviewed at home. Medications, including use of cholesterol-lowering agents either currently (or in the previous 5 years), smoking, history of cardiovascular disease, and main (or partner's) occupation were recorded. Participants were invited to a clinic at the Northern General Hospital, Sheffield, United Kingdom. 392 (95% of those interviewed) attended, and stereoscopic photographs of both fundi were taken.

Photographs were graded by one observer (NH) unaware of the participants' drug history, against standard images using the Wisconsin Age Related Maculopathy Grading System (WARMGS; Klein et al, 1991, Ophthalmology 98, 1128-1134). 12 participants who had non age-related degenerative macular changes, and one who was taking part in a trial of statins, were excluded. The analyses presented are therefore based on 379 participants.

Of the 379 subjects, 27 (7.1 %) reported use of statins, and 77 (20.3%) had some evidence of macular degeneration.

### Example 2

Statistical Analysis

Firstly, the number and percentage of subjects with macular degeneration in either eye is described according to use of statins (coded no or yes). The first analyses used three definitions which distinguish between early and late stages of macular degeneration (a) early only, (b) late only, (c) early/late combined; these definitions are those as set out above in the section “Features of Normal, Early and Late Macular Degeneration”. For each definition, Fisher's exact test is used to test whether the proportion of subjects with macular degeneration is significantly different in non-users compared with users of statins. The p-value for Fisher's exact test is presented in each case.

Secondly, logistic regression modelling was used to calculate the odds ratio (and 95% confidence interval) for early/late macular degeneration combined among subjects not using statins compared with those using statins (the reference category). Odds ratios are calculated without adjustment for any potential confounders, and then with adjustment for gender, age, and history of coronary artery bypass grafting or angioplasty.

### Example 3

Results

TABLE 1
Table 1 classifies participants by type of
macular degeneration and stain use.

| | Use of Statins Currently or | | |
|---|---|---|---|
| Type of age-related macular | in the Last 5 Years | | |
| degeneration* | No | Yes | Total |
| None | 276 (78.4) | 26 (96.3) | 301 (79.7) |
| Early only (excluding late) | 62 (18.3) | 1 (3.7) [symbol] | 63 (17.3) |
| Late only (excluding early) | 14 (4.8) | 0 (0) | 14 (4.4) |
| Combined (early or late) | 76 (21.6) | 1 (3.7)^{⊕} | 77 (20.3) |

Numbers (%) of participants with age-related macular degeneration in either eye by use of statins.
*The presence of early and late age-related macular degeneration was determined by WARMGS criteria according to the level of degeneration in the worse eye. [symbol]
X² = 3.75; p = 0.053
^{⊕}X² = 5.0; p = 0.026.

Age-related macular degeneration was more frequent among participants who did not take statins. 76 out of 352 (21.6%) of participants who did not take statins showed signs of early or late macular degeneration, compared to only 1 out of 27 (3.7%) of participants taking statins who showed signs of the disease (χ²=5.0; p=0.03). this is equivalent to an odds ratio for macular degeneration among participants who took statins of 0.14 (95% CI 0.02 -0.83) compared with those who did not. None of the 27 participants who had been taking statins had late age-related macular degeneration compared to 14 out of 352 (4.0%) participants who had not taken the drug (p=0.29). early macular degeneration was found in only one participant among 27 (3.7%) who had been taking statins but in 62 of the 338 (18.3%) participants who had not been taking the drug (χ²=3.8; p=0.053).

A history of coronary artery bypass grafting or angioplasty was associated with macular degeneration. 9 out of the 78 participants with macular degeneration (12%) had undergone coronary angioplasty or bypass grafting compared with 13 out of the 302 participants (4%) without macular degeneration (χ²=5.9; p=0.01). Not surprisingly, people who had undergone coronary angioplasty or bypass grafting were more likely to have taken statins (6 out of 22=27% compared with 21 out of 389=5% non-users of the drug). In a regression model, after adjusting for age, sex, smoking, social class, and a history of coronary angioplasty or bypass grafting, the odds ratio for macular degeneration (early or late) among participants taking statins was 0.09 (95% CI0.01-0.72).

### Example 4

A Case-Control Study of the Influence of Statins on the Risk of Macular Degeneration in Elderly People

A retrospective case-control study is conducted to confirm the above results. The case-control study is conducted at the Southampton Eye Hospital, Southampton University NHS Hospitals Trust.

All people diagnosed has having age-related macular degeneration by consultant ophthalmologists at the Southampton Eye Hospital are eligible as cases. It is expected that, over a year, it is possible to recruit around 200 cases, allowing for a 40% refusal rate.

Two groups of controls are employed. The first, hospital-based controls, are recruited from patients in the same age range as cases attending the Eye Hospital. Three controls are recruited per case in order to maximise statistical power. A second control group is recruited from the community: general practitioners are asked to select the three patients closest in age and of the same sex as the case on his or her list.

Cases and controls are approached for permission for the investigators to view their general practice records. Those who agree have their records scrutinised and a detailed drug history dating back to five or six years (e.g., to 1995) is recorded. Information, where available, is also be obtained on age, smoking habit and presence of, or treatment for, symptomatic cardiovascular disease and hypertension.

In the group under study, around 10% of subjects have been taking statins over the time period. The study is designed to have adequate statistical power to detect a 50% reduction in risk of macular degeneration associated with a history of taking statins for more than six months with ∀0.05 and ∃0.8.

Statistical analysis of the association between statin use and macular degeneration is carried out by logistical regression techniques, as described in detail below. It is found that patients undergoing treatment with statins show a significantly reduced risk of developing macular degeneration.

A cohort study is also carried out. The results of the cohort study confirm the case-control study.

### Example 5

Randomised Controlled Trial of Statins in the Prevention of Progression of Early Age-Related Macular Degeneration

The above Examples show that that people taking drugs in the statin class of lipid lowering agents are at reduced risk of developing age-related macular degeneration. A clinical trial is undertaken to confirm that statins slow down or halt the pathogenic processes occurring in the retina that lead to macular degeneration.

The clinical trial employed is a randomised, parallel group, double-masked, placebo-controlled trial. The trial is conducted on a group of patients with early stage of age-related macular degeneration.

The principal outcome is the amount of progression of macular degeneration, as judged by the Wisconsin Age-Related Macular Degeneration Score, after three months, six months and one year's treatment. Secondary outcomes will be visual acuity and health-related quality of life.

Cases are recruited from ophthalmology departments at several centres in the UK. Eligibility criteria include age between 65 and 75, diagnosis of early age-related macular degeneration by a consultant ophthalmologist, patient not currently taking lipid lowering drugs, absence of contraindications to taking HMG CoA reductase inhibitors—in particular, active liver disease or a history of alcohol abuse.

Patients are randomised either to 10 mg of simvastatin each night or to placebo. Plasma lipid concentrations are measured at baseline and at 3, 6 and 12 months, but the results are not made available to those involved with the clinical care of the patients. The Wisconsin Age-Related Macular Degeneration Score, corrected visual acuity, and health-related quality of life are also measured at baseline at 3, 6 and 12 months. Analysis is on an intention-to-treat basis with the primary outcome as the Wisconsin Age-Related Macular Degeneration Score.

It is found that patients who are given simvastatin show improved Macular Degeneration Score as compared to patients who are given the placebo. In addition, the former group shows improvement in visual acuity. The results are statistically significant

### Example 6

Detailed Explanation of Statistical Analysis

Regression is a statistical technique in which a model is estimated that best explains the relation between an outcome variable Y and a predictor variable X. The regression model is estimated from data that has been collected about X and Y for a sample of n observations/subjects. Commonly, the outcome variable Y is continuously distributed, e.g. blood pressure, weight.

Logistic regression is a regression method used for modelling binary (yes/no) outcome variables e.g. 0=alive and 1=dead, or 0=no macular degeneration and 1=early/late macular degeneration combined. The interest is in modelling the proportions of subjects experiencing the event in question in relation to our predictor variable e.g. modelling the proportions of subjects with early/late macular degeneration according to use of statins. The proportions cannot be modelled directly and instead the relation between the predictor variable, e.g. category of statin use, and the loge transformation of the odds of the event is modelled. The “odds of an event” are defined as p/(1−p) where p is the probability of the event in question (in other words, the odds of an event is the probability of the event occurring divided by the probability of the event not occurring). This approach prevents us obtaining a model that predicts impossible values for a proportion i.e. outside the range 0 to 1.

Results from logistic regression models are commonly presented as odds ratios (OR). An odds ratio is simply the ratio of two odds. For example, in the present study, we compare the odds of early/late macular degeneration in subjects who did not take statins with those who did take statins as a reference category. In this way, we obtain the odds ratio for early/late macular degeneration in non-users of statins compared with users of statins. If the odds ratio is 1, this means that the odds of the event are estimated to be the same in the group of interest and the reference group. If the odds ratio is greater than 1 this means that the odds of the event is greater in the group of interest than in the reference group. Conversely, if the odds ratio is less than 1 this means that the odds of the event are smaller in the group of interest than in the reference group.

Logistic regression has the advantage that associations between X and Y may be investigated after accounting for whether X and Y may simply appear to be associated because of their both being associated with another variable Z. A variable Z that is associated with both X, and with Y, is referred to as a as a potential confounder of the association between X and Y. It may be the case that failure to account for Z will obscure a true association between X and Y (negative confounding by Z) or it may be the case that failure to account for Z will give rise to a spurious association between X and Y (positive confounding by Z). The potential confounding effect of Z may be accounted for, or adjusted, by including Z in the logistic regression model of Y on X. This is what is meant by saying that the macular degeneration odds ratios are “adjusted for age, gender and history of coronary artery bypass grafting or angioplasty”. In other words, the odds ratios have been calculated after taking into account the possible confounding effect of Z (age, gender and history of coronary artery bypass grafting or angioplasty) on the relation between X (use of statins) and Y (early/late macular degeneration).

Typically, the odds ratio alone is not presented. Rather, the odds ratio is associated with a 95% confidence interval to reflect the uncertainty associated with the estimate of the odds ratio. A confidence interval (CI) is a range of values within which the ‘true’ odds ratio is believed to be found, with a given level of confidence. An odds ratio is estimated to be 1.5 but a 95%CI from 1.3 to 1.7 reflects the fact that it is 95% certain that the true odds ratio is found within the range 1.3 to 1.7. The rationale for calculating confidence intervals is the uncertainty which is always associated with using samples to obtain information about a broader population from which the sample is selected. A single value estimate is likely to be inaccurate and so a confidence interval provides additional information about the true population value.

A p-value is used to assess the evidence in the data for an alternative hypothesis versus a null hypothesis. The null hypothesis is set up as the hypothesis of no change/no difference/no effect.

For example, in this study, the null hypothesis may be set up that there is no difference in the odds of early/late macular degeneration in non-users compared with users of statins, i.e. the odds ratio is 1. The 2-sided alternative hypothesis is that the odds ratio differs from 1. The p-value associated with the hypothesis test is calculated, where the p-value is the probability of observing what you have observed, or an outcome more extreme, given that the null hypothesis were true. The p-value may also be regarded as the probability of observing what has been observed simply by chance. Smaller p-values indicate stronger evidence against the null hypothesis. For example, if a p-value is 0.004 this is interpreted as saying that there is only a 4 in 1000 chance of observing what has been observed, or a more extreme scenario, when truly there is no real effect, i.e. when truly the null hypothesis applies. Conventionally, a result is said to be statistically significant at the 5% level if p≦0.05.

Fisher's exact test is a test for comparing proportions between independent groups. The null hypothesis is that there is no difference in proportions between the groups.

Statistical definitions are adapted from: A-Z of Medical Statistics, a companion for critical appraisal. Filomena Pereira-Maxwell. Arnold Publishers, 1998.

### Example 7

The Wisconsin Age Related Maculopathy Grading System (WARMGS)

The Wisconsin Age Related Maculopathy Grading System is described in detail in Klein et al, 1991, Ophthalmology 98, 1128-1134. An outline of the grading system is provided here.

Stereoscopic photographic pairs centered on the disc and macula (fields 1 and 2 of the modified Airlie House classification (Diabetic Retinopathy Study Research Group. Report 7. A modification of the Airlie House classification of diabetic retinopathy. Invest Ophthalmol Vis Sci 1981, 21:210-26)) are mounted in clear plastic sheets and placed on a fluorescent viewing box furnishing light with a Kelvin rating of approximately 6200°. Graders examine the slides with Donaldson stereoscopic viewers, which provide 5× magnification. Combined with the approximately 3× magnification provided by the fundus camera, this results in a total magnification of 15×.

Before grading, a grid consisting of three circles concentric with the center of the macula and four radial lines is superimposed over one member of the stereoscopic pair of field 2. The radius of the innermost circle corresponds to 500 μm in the fundus of an average eye and the radii of the middle and outer circles to 1500 μm and 3000 μm, respectively.

Nine subfields are defined by the grid: the central subfield (within the inner circle); the inner superior, inner nasal, inner inferior, and inner temporal subfields (between the inner and middle circles); and the outer superior, outer nasal, outer inferior, and outer temporal subfields (between the middle and outer circles). Some characteristics are graded in each subfield, others in field 2 as a whole. Three sets of open circles printed on clear plastic are used to estimate size of drusen, area involved by drusen, and area involved by increased pigmentation, as described in a later section.

The grading system is organized into three sections, the first dealing with various characteristics of drusen, the second with other lesions typical of ARMD, and the third with other abnormalities. Written definitions are used for most steps in the grading scales. A set of stereoscopic photographs is used to illustrate the appearances described in a detailed written protocol. The protocol is set out in detail in a document from the National Technical Information Service (Klein R, Davis M D, Magli Y L, Klein B E K. Wisconsin Age-Related Maculopathy Grading System. Madison, Department of Ophthalmology,, University of Wisconsin School of Medicine 1991 US Dept. of Commerce. Available from: National Technical Information Service, 5285 Port Royal Rd, Springfield, Va. 22161 Accession #PB91-184257/AS.)

In WARMGS, four characteristics of drusen are graded in semiquantitative fashion in each subfield: maximum size, predominant type, area, and degree of confluence. Predominant drusen size and the maximum extent of confluence are also evaluated for all of field 2 as a whole. Standard circles C₀, C₁, and C₂, with diameters corresponding to 63 μm, 125 μm, and 250 μm, are used for estimating maximum drusen size and predominant drusen type. These two characteristics represent slightly different approaches to describing the appearance of drusen and are thus closely correlated.

Drusen size. In each subfield, the largest drusen present determines the grade for maximum drusen size, on the following scale:

| Grade | Definition |
|---|---|
| 0 | No drusen |
| 1 | Drusen indistinct or questionable, or stippling |
| 2 | Drusen distinct, but diameter < circle C₀ (63 μm) |
| 3 | Drusen ≧ circle C₀ but < C₁ in diameter (63 to 124 μm) |
| 4 | Drusen ≧ circle C₁ but < circle C₂ in diameter (125 to 249 μm) |
| 5 | Drusen ≧ circle C₂ in diameter (≧250 μm) |
| 6 | Reticular drusen |
| 7 | Cannot grade—obscuring legion |
| 8 | Cannot grade—photographic quality |

Drusen type. In each subfield, the type of drusen occupying the largest fraction of the subfield determines the grade for predominant drusen type, with type based on the size of drusen, the uniformity of appearance across their breadth, and the sharpness of their edges. The following terms are used:

| Grade | Definition |
|---|---|
| 0 | Hard-indistinct |
| 1 | Hard-distinct |
| 2 | Soft-distinct |
| 3 | Soft-indistinct |
| 4 | Reticular |
| 5 | Faded |
| 6 | Cannot grade—obscuring lesion |
| 7 | Cannot grade—photographic quality |

Drusen less then 63 μm in diameter are classified as hard, drusen between 63 and 124 μmdiameter may be placed in the hard distinct or either of the soft categories, and drusen 125 μm in diameter or greater are classified as soft. There are no specific size requirements for reticular or faded drusen, which usually are at least 125 μm in diameter. Small drusen are separated into two subtypes: hard-indistinct and hard-distinct. Hard-distinct drusen (grade 2 or 3 for drusen size, grade 1 for drusen type) are unequivocal. The hard-distinct drusen shows little variation.

Lesions believed to be drusen but that appear less definite are placed in the hard-indistinct category (grade 0). Also placed in this category are lesions that the grader believes to be drusen with at least 50% but less than 90% certainty (questionable), and lesions with the appearance designated stippling described below. Subfields with stippling (with or without hard-indistinct or questionable drusen, but free of hard-distinct or larger drusen) are graded 1 for drusen size, 0 for drusen type. Drusen as large as or larger than circle C₀ but smaller than circle C₁ are placed in either a hard, soft, or faded category on the basis of uniformity of density from center to periphery, sharpness of edges, and thickness, as described below. Drusen with decreasing density from center to periphery and fuzzy edges are generally placed in the soft-indistinct category; those with uniform density, sharp edges, and a solid, thick appearance are placed in the soft-distinct category; those with sharp edges but without a solid, thick, nodular appearance are placed in the hard-distinct category; and those in which virtually all of the drusen substance appears to have disappeared are placed in the faded category. Faded drusen are distinguished from RPE degeneration by the round or oval shape of the former and their separation from one another. If patches of highly confluent druscn were to bccomc faded, they would be graded as RPE degeneration. Drusen as large as or larger than circle C, are placed in one of the soft categories or in the reticular or faded categories. In this classification, the term “reticular” is used to describe drusen that form ill-defined networks of broad interlacing ribbons.

Drusen area. Drusen area is estimated for each subfield in which drusen size is grade 2 or greater The area covered by drusen within each subfield is estimated by mentally moving together all dmsen graded at least 2 for size as if they were confluent and comparing this area with the areas of the standard circles. For the lower steps in the scale, circles C₁ and C₂ are used in each subfield. For higher steps, circles 1 and 2 specific for the subfield being graded are used (C₁ and C₂ for the central subfield, I₁ and I₂ for the inner subfields, and O₁ and O₂ for the outer subfields). The circles specific to each subfield represent a fraction of that subfield: C₁, I₁, and O₁ are 1/64 or 1.6% of the central, inner, and outer subfields respectively; C₂, I₂, and O₂ are 1/16 or 6.3% of the same subfields. The grading scale is shown below.

| Grade | Definition |
|---|---|
| N/A | Not applicable (i.e., no drusen ≧ grade 2 for size) |
| 0 | Area covered by drusen < C₀ |
| 1 | Area covered by drusen < C₁* |
| 2 | Area covered by drusen < circle 1 (<1.6% of the subfield)* |
| 3 | Area covered ≧ circle 1, but < twice this area (≧1.6% but < |
| | 3.1% of subfield) |
| 4 | Area covered ≧ twice circle 1, but < circle 2 (≧3.1% but < |
| | 6.3% of subfield) |
| 5 | Area covered ≧ circle 2, but < twice this area (≧6.3% but < |
| | 12.5% of subfield) |
| 6 | Area covered ≧ twice, but < four times circle 2 (≧12.5% but < |
| | 25% subfield) |
| 7 | Area covered ≧ 25% of subfield, but < 50% |
| 8 | Area covered ≧ 50% of subfield |

Drusen confluence. Drusen confluence is defined as any touching or merging of two or more drusen. Confluence is graded in each subfield in which maximum drusen size is grade 2 or greater according to the percent of total drusen area involved, using the following scale: none, questionable or less than 10%, 10% to 24%, 25% to 49%, and 50% or more. *(For the central subfield, grades 1 and 2 are equivalent and are indicated on the grading form as 1/2.)

Several other characteristics of age-related macular degeneration are assessed in each subfield: presence and extent of RPE degeneration, increased pigment, RPE detachment and/or serous detachment of the sensory retina (SSR), retinal hard exudate, subretinal and/or sub-RPE hemorrhage, subretinal and/or sub-RPE fibrous tissue, geographic atrophy, retinal edema (thickening), and retinal hemorrhages.

Retinal pigment epithelium degeneration is estimated as a percentage of the subfield, using the following categories none, questionable, less than 25% of subfield, 25% to 49% of subfield, 50% or more of subfield, and cannot grade.

The presence and severity of granules or clumps of gray or black pigment in or beneath the retina are also graded. The grading scale is: none, questionable, area less than circle C₂, area greater than or equal to circle C₂, cannot grade because of hyperpigmentation from non-ARMD lesions, and cannot grade for other reasons.

Because it is often difficult to distinguish between RPE and SSR detachment without fluorescein angiography and because RPE and SSR often occur together, they are assessed in a single item of the classification. Separate codes are used to designate detachments that are mostly RPE, mostly SSR, or a mixture of both. The extent of RPE/SSR detachment within the 16-disc area zone covered by the grid is estimated in disc areas in a separate item.

Subretinal and sub-RPE hemorrhages, graded as one lesion in each subfield, are designated as absent, questionable, present, or cannot grade.

Sheets or mounds of white material under the retina in eyes with ARMD usually represent fibrous or fibro-vascular tissue. The grades for subretinal fibrous scar are: absent, questionable, less than 25% of subfield, 25% to 49% of the subfield, 50% or more of the subfield, and cannot grade. No attempt is made to distinguish between fibrin and fibrous tissue in the classification.

Geographic atrophy is a sharply defined area of dropout of the RPE, exposing choroidal blood vessels. For purposes of this grading scheme, an area of atrophy must be at least as large as standard circle I₁ to be considered definitely present. The grading scale is: absent, questionable, less than 50% of subfield, 50% or more of subfield, and cannot grade.

Chorioretinal scars from past trauma, from a congenital process, or from other causes are designated present under the heading “other lesions.”

Each of the applications and patents mentioned above, and each document cited or referenced in each of the foregoing applications and patents, including during the prosecution of each of the foregoing applications and patents (“application cited documents”) and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of treating or preventing macular degeneration in a patient, the method comprising administering an HMG-CoA reductase inhibitor to the patient.

2. A method of treating or preventing macular degeneration in a patient, the method comprising reducing the activity of an HMG-CoA reductase enzyme in a patient.

3. A method of using an HMG-CoA reductase inhibitor for the preparation of a pharmaceutical composition for the treatment or prevention of macular degeneration in a patient comprising:
(a) admixing said HMG-CoA reductase inhibitor with a pharmaceutically acceptable carrier so as to generate a pharmaceutical composition; and
(b) administering said pharmaceutical composition to said patient.

4. The method of claim 1, 2, or 3, in which the HMG-CoA reductase inhibitor comprises a statin.

5. The method of claim 4, in which the HMG-CoA recductase inhibitor comprises a statin selected from the group consisting of: fluvastatin (Lescol), cerivastatin (Baycol), atorvastatin (Lipitor), simvastatin (Zocor), pravastatin (Pravachol), lovastatin (Mevacor) and rosuvastatin (ZD 4522).

6. A method of treating or preventing macular degeneration in a patient, the method comprising one or more of the following:
(a) lowering the level of LDL cholesterol in the patient;
(b) increasing the level of HDL cholesterol in the patient; and
(c) lowering the level of triglycerides in the patient.

7. The method of claim 6, in which the level of LDL cholesterol is reduced by at least 20%, or in which the level of HDL cholesterol is increased by at least 5%, or in which the level of triglycerides is reduced by at least 10%.

8. The method of claim 1, 2, 3, or 6, in which the incidence or progression of macular degeneration is prevented in a patient.

9. The method of claim 1, 2, 3, or 6, in which the patient exhibits one or more of the following: (i) reduced accumulation of basal linear deposit in Bruch's membrane; (ii) protection of the outer retina from oxidative damage; and (iii) inhibition of endothelial cell apoptosis.

10. The method of claim 1, 2, 3, or 6, in which at least one of the following symptoms is reduced, eliminated, or prevented from developing or progressing in a patient: deterioration in sight, loss or reduction of visual acuity, loss of vision, distortion of vision, loss of central vision, increased macular pigment, presence of drusen, progression of small drusen to large drusen, progression of small drusen to confluent drusen, choroidal neovascularisation, progression of early disease to late, progression from dry to wet form or vice versa, geographic atrophy, RPE degeneration, RPE detachment, serous detachment of the sensory retina (SSR), retinal hard exudate, subretinal and/or sub-RPE haemorrhage, subretinal and/or sub-RPE fibrous tissue, retinal edema (thickening) and retinal haemorrhage.

11. The method of claim 1, 2, 3, or 6, in which macular degeneration in a second eye is prevented from developing or progressing in a patient having macular degeneration in one eye.

12. The method of claim 1, 2, 3, or 6, in which the age-related macular degeneration comprises early macular degeneration.

13. The method of claim 1, 2, 3, or 6, in which the age-related macular degeneration comprises late macular degeneration.

14. The method of claim 1, 2, 3, or 6, in which progression of early to late age-related macular degeneration is prevented in the patient.

15. A method of predicting whether a patient is likely to develop macular degeneration, the method comprising determining whether the patient is suffering from, or has suffered from, stroke or coronary disease, in which if the patient has suffered or is suffering from stroke or coronary disease, then the patient is likely to develop macular degeneration.

16. The method of claim 15, in which the patient is considered as suffering or having suffered from coronary disease if said patient is undergoing or has undergone one or more of the following: coronary bypass grafting; heart by-pass surgery; angioplasty; a heart attack; coronary artery disease and claudication.
